(19) United States
(12) Patent Application Publication
(10) Pub. No.: US 2003/0068643 A1
(43) Pub. Date: Apr. 10, 2003

(54) Methods and compositions for economically synthesizing and assembling long DNA sequences

(76) Inventors: Thomas M. Brennan, San Francisco, CA (US); Herbert L. Heyneker, San Francisco, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/298,081

(22) Filed: Nov. 14, 2002

Related U.S. Application Data
(63) Continuation of application No. 09/264,388, filed on Mar. 8, 1999, now abandoned.

(51) Int. Cl. C12Q 1/68; C12P 19/34

(52) U.S. Cl. 435/6; 435/91.2

(57) ABSTRACT

The present invention relates to a method for synthesizing and assembling long DNA sequences from short synthetic oligonucleotides. More specifically, the present invention is a cost-effective method for producing large segments of DNA of interest.

### FIELD OF THE INVENTION

The present invention relates to a method for synthesizing and assembling long DNA sequences from short synthetic oligonucleotides. More specifically, the present invention is a cost-effective method for producing large segments of DNA of interest.

### BACKGROUND OF THE INVENTION

The advent of rapid sequencing technology has created large databases of DNA sequences containing useful genetic information. The remaining challenges are to find out what these genes really do, how they interact to regulate the whole organism, and ultimately how they may be manipulated to find utility in gene therapy, protein therapy, and diagnosis. The elucidation of the function of genes requires not only the knowledge of the wild type sequences, but also the availability of sequences containing designed variations in order to further the understanding of the roles various genes play in health and diseases. Mutagenesis is routinely conducted in the laboratory to create random or directed libraries of interesting sequence variations. However the ability to manipulate large segments of DNA to perform experiments on the functional effects of changes in DNA sequences has been limited by the enzymes available and their associated costs. For example, the researcher cannot easily control the specific addition or deletion of certain regions or sequences of DNA via traditional mutagenesis methods, and must resort to the selection of interesting DNA sequences from libraries containing genetic variations.

It would be most useful if a researcher could systematically synthesize large regions of DNA to determine the effect of differences in sequences upon the function of such regions. However, DNA synthesis using traditional methods is impractical because of the declining overall yield. For example, even with a yield of 99.5% per step in the phosphoramidite method of DNA synthesis, the total yield of a full length sequence of 500 base pairs long would be less than 1%. Similarly, if one were to synthesize overlapping strands of, for example, an adenovirus useful as a gene therapy vector, the 50-70 kilobases of synthetic DNA required, even at a recent low price of approximately $1.00 per base, would cost over $50,000 per full sequence, far too expensive to be practical when compared with the enzymatic synthesis of DNA using PCR technology.

The recovery of long segments of DNA by chemical synthesis may be improved when the DNA chemical synthesis is combined with recombinant DNA technology. Goeddel et al., Proc. Natl. Acad. Sci. USA 76(1):106-110 (1979); Itakura et al., Science 198:1056-1063 (1977); and Heyneker et al., Nature 263:748-752 (1976). The synthesis of a long segment of DNA may begin with the synthesis of several modest-sized DNA fragments by chemical synthesis and continue with enzymatic ligation of the modest-sized fragments to produce the desired long segment of DNA. Synthetically made modest-sized DNA fragments may also be fused to DNA plasmids using restriction enzymes and ligase to obtain the desired long DNA sequences, which may be transcribed and translated in a suitable host. Recently, self-priming PCR technology has been used to assemble large segments of DNA sequences from a pool of overlapping oligonucleotides by using DNA polymerase without the use of ligase. Dillon et al., BioTechniques 9(3):298-300 (1990); Prodromou et al., Protein Engineering 5(8):827-829 (1992); Chen et al., J. Am. Chem. Soc. 116:8799-8800 (1994); and Hayashi et al., BioTechniques 17(2):310-315 (1994). Most recently, DNA shuffling method was introduced to assemble genes from random fragments generated by partial DNAaseI digestion or from a mixture of oligonucleotides. Stemmer, Nature 370:389-391 (1994) and Stemmer, Proc. Natl. Acad. Sci. USA 91:10747-10751 (1994).

Methods to synthesize a large variety of short or modest-sized oligonucleotides have been extensively described. One of the methods is to use microarray technology, where a large number of oligonucleotides are synthesized simultaneously on the surface of glass or DNA chips. The microarray technology has been described in U.S. Pat. Nos. 5,510,270, 5,412,087, 5,445,934, 5,744,305, 5,843,655, and 5,807,522, which are incorporated herein by reference. Typically, high density arrays of DNA fragments are fabricated on glass or nylon substrates by in situ light-directed combinatorial synthesis or by conventional synthesis followed by immobilization. However, this photolithography synthesis method provides neither oligonucleotides which are pure enough for later enzymatic assembly nor a method which is flexible and cost effective. For example, several hundreds of thousands of dollars of masks specific to any given series of sequences are required for practical assembly. Another method for synthesizing DNA in arrays has been described by Brennan, U.S. Pat. No. 5,474,796, incorporated herein by reference. A high-yield, chip-based synthesis is achieved using ink-jet like dispensers to deposit different reagents for DNA synthesis at different functionalized binding sites on the surface of a DNA synthesis substrate. In such a system, the linker which binds the DNA under construction to the surface can be made cleavable from the surface under conditions remote from the DNA synthesis conditions. Such a linker allows a large number of different oligonucleotides to be synthesized separately, and cleaved to form a mixture in solution of the oligonucleotide sequences for assembly into a longer DNA sequence. Such technology, including the ability to perform cleavable chip based custom synthesis, is required to make synthesis and assembly economically practical.

Existing methods for the synthesis of long DNA sequences have many drawbacks, for example, the length limitations of conventional solid phase DNA synthesis, the requirement of synthesizing both strands of DNA, the complexity of multiple enzymatic reactions, the necessity of purifying a large number of oligonucleotides. These drawbacks inevitably add to the cost of obtaining long DNA sequences. There is a need in the art to economically synthesize and assemble large segments of DNA sequence. Such an inexpensive and custom synthesis and assembly process has many uses. Gene sequences of interest, much larger than can be synthesized via traditional controlled pore glass (CPG) or DNA chip-based synthesis, can be assembled and tested for a variety of functionality, for example, the function of relative position of promoter to gene coding sequence, the role of introns versus exons, the minimization of gene sequence necessary for function, the role of polymorphisms and mutations, the effectiveness of sequence changes to gene therapy vectors, the optimization of a natural protein for a specific experiment or industrial application, among others. These functional analysis may be explored with the DNA designs truly under the control of the researcher. In other cases, specific variations in assembled sequence can be used to create structured libraries containing many possible genetic variations for testing of the function or the inhibition of the function. Eventually entire genomes could be easily synthesized, assembled, and functionally tested in this manner. In short, any experiment in which a model system of functioning nucleotides could be changed in a specific way under the control of a researcher, could be performed easily and less expensively.

### SUMMARY OF THE INVENTION

The present method for synthesizing and assembling long DNA sequences from synthetic oligonucleotides comprises the steps of: (a) attaching a cleavable linker to an oligonucleotide synthesis substrate; (b) designing overlapping oligonucleotide sequences, which collectively encode both strands of the target DNA, cDNA or RNA sequence; (c) synthesizing an array of chosen oligonucleotide sequences on the surface of a substrate; (d) cleaving the synthesized oligonucleotide sequences from the surface of the array substrate to form a mixture of the overlapping oligonucleotides; and (e) assembling the mixture of overlapping oligonucleotides into the target full-length sequence.

In particular, the cleavable linker may be detached from the oligonucleotide synthesis substrate without damaging the synthesized oligonucleotides. The array of overlapping oligonucleotides may be synthesized simultaneously. Preferably, the simultaneous synthesis of the overlapping oligonucleotides may be carried out according to the method disclosed in Brennan, U.S. Pat. No. 5,474,796, incorporated herein by reference. The target long DNA sequences contemplated in the present invention may be a regulatory sequence, a gene or a fragment thereof, a vector, a plasmid, a virus, a full genome of an organism, or any other biologically functional DNA sequences which may be assembled from overlapping oligonucleotides, either directly or indirectly by enzymatic ligation, by PCR-based technology, or by other suitable assembly methods known in the art.

### BRIEF DESCRIPTION OF THE FIGURES

FIG. 1 shows hydroxyl-group bearing non-cleavable linkers used for hybridization directly on the glass chip.

FIG. 2 shows the coupling of a chemical phosphorylation agent as the special amidite to allow cleavage of the oligonucleotide after synthesis.

FIG. 3 shows the amidite (TOPS) used to prepare universal CPG-support to allow cleavage of the oligonucleotide after synthesis.

FIG. 4A illustrates the formation of an array surface that is ready for solid phase synthesis.

FIG. 4B illustrates O-Nitrocarbamate array making chemistry.

FIG. 5 illustrates surface tension wall effect at the dot-interstice interface. The droplet containing solid phase synthesis reagents does not spread beyond the perimeter of the dot due to the surface tension wall.

FIG. 6 illustrates hydrogen-phosphonate solid phase oligonucleotide synthesis on an array surface prepared according to Example 1.

FIG. 7 illustrates top and side views of a piezoelectric impulse jet of the type used to deliver solid phase synthesis reagents to individual dots in the array plate synthesis methods according to the invention.

FIG. 8 illustrates use of a piezoelectric impulse jet head to deliver blocked nucleotides and activating agents to individual dots on an array plate. The configuration shown has a stationary head/moving plate assembly.

FIG. 9 illustrates an enclosure for array reactions showing array plate, sliding cover and manifolds for reagent inlet and outlet.

### DETAILED DESCRIPTION OF THE INVENTION

The present invention relates to a method for synthesizing and assembling long DNA sequences from short synthetic oligonucleotides. More specifically, the present invention is a cost-effective method for producing large segments of DNA of interest.

In general, the present method for synthesizing and assembling long DNA sequences from synthetic oligonucleotides comprises the steps of: (a) attaching a cleavable linker to an oligonucleotide synthesis substrate; (b) designing overlapping oligonucleotide sequences, which collectively encode both strands of the target DNA, cDNA or RNA sequence; (c) synthesizing an array of chosen oligonucleotide sequences on the surface of a substrate, (d) cleaving the synthesized oligonucleotide sequences from the surface of the array substrate to form a mixture of the overlapping oligonucleotides, and (e) assembling the mixture of overlapping oligonucleotides into the target full-length sequence. In particular, the cleavable linker may be detached from the oligonucleotide synthesis substrate without damaging the synthesized oligonucleotides. In addition, the array of overlapping oligonucleotides may be synthesized simultaneously. The target long DNA sequences contemplated in the present invention may be a regulatory sequence, a gene or a fragment thereof, a vector, a plasmid, a virus, a full genome of an organism, or any other biologically functional DNA sequences which may be assembled from overlapping oligonucleotides, either directly or indirectly by enzymatic ligation, by PCR based technology, or by other suitable assembly methods known in the art.

Attachment of a Cleavable Linker to a Substrate

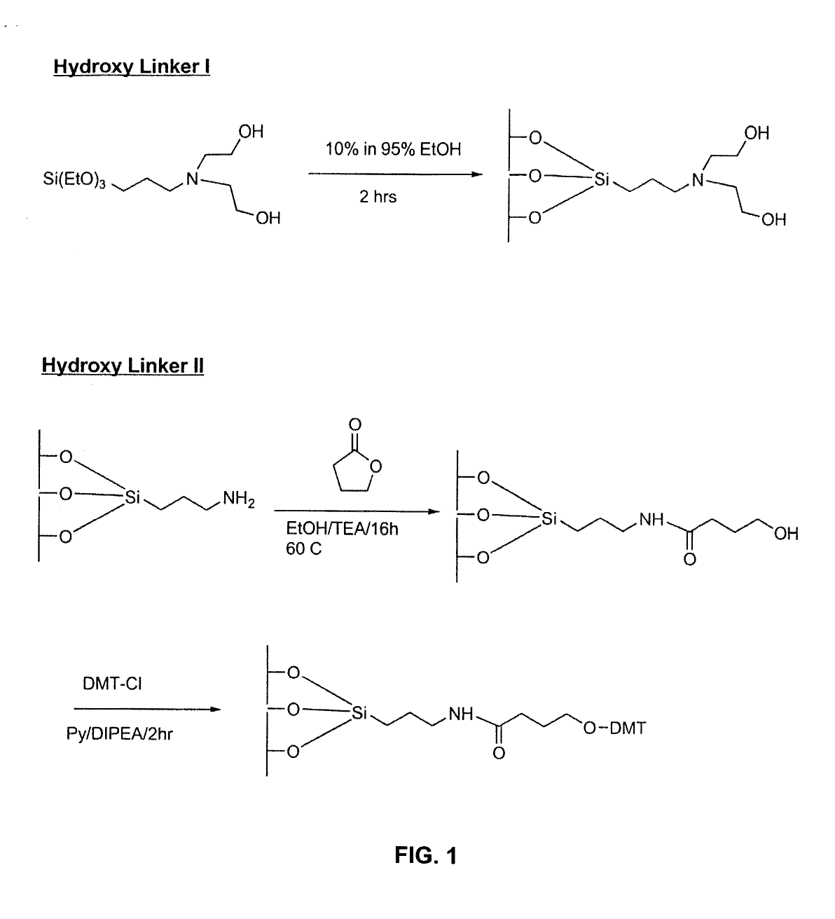

In solid phase or microarray oligonucleotide synthesis designed for diagnostic and other hybridization-based analysis, the final oligonucleotide products remain attached to the solid support such as controlled-pore glass (CPG) or chips. A non-cleavable linker such as the hydroxyl linker I or II in FIG. 1 is typically used. These hydroxyl linkers remain intact during the deprotection and purification processes and during the hybridization analysis. Synthesis of a large number of overlapping oligonucleotide for the eventual assembly into a longer DNA segment, however, must be performed on a linker which allows the cleavage of the synthesized oligonucleotide. The cleavable linker is removed under conditions which do not degrade the oligonucleotide. Preferably the linker may be cleaved using two approaches, either under the same conditions as the deprotection step or utilizing a different condition or reagent for linker cleavage after the completion of the deprotection step. The former approach may be advantageous, as the cleavage of the linker is performed at the same time as the obligatory deprotection of the nucleoside bases. Time and effort are saved to avoid additional post-synthesis chemistry. The cost is lowered by using the same reagents for deprotection in the linker cleavage. The second approach may be desirable, as the linker cleavage may serve as a pre-purification step, eliminating all protecting groups from the solution used for subsequent assembly and amplification.

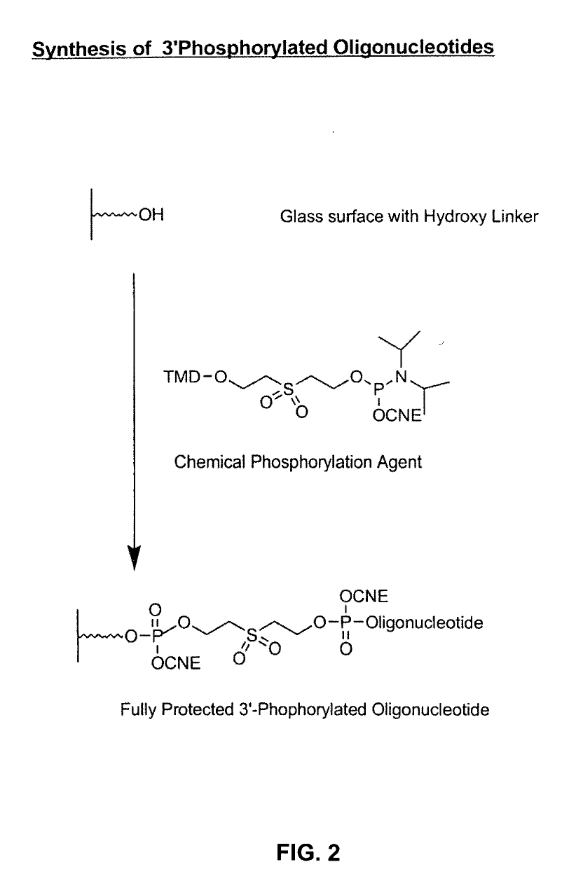

A broad variety of cleavable linkers are available in the art of solid phase and microarray oligonucleotide synthesis. Pon, R., “Solid-Phase Supports for Oligonucleotide Synthesis” in “Protocols for oligonucleotides and analogs; synthesis and properties,” Methods Mol. Biol. 20:465-496 (1993). A suitable linker may be selected to be compatible with the nature of the protecting group of the nucleoside bases, the choice of solid support, the mode of reagent delivery, among others. For example, in order to convert the non-cleavable hydroxyl linker (FIG. 1) into a cleavable linker, a special phosphoramidite may be coupled to the hydroxyl group prior to the phophoramidite or H-phosphonate oligonucleotide synthesis. One preferred embodiment of such special phophoramidite, a chemical phosphorylation agent, is shown in FIG. 2. The reaction conditions for coupling the hydroxyl group with the chemical phosphorylation agent are known to those skilled in the art. The cleavage of the chemical phosphorylation agent at the completion of the oligonucleotide synthesis yields an oligonucleotide bearing a phosphate group at the 3′ end. The 3′-phosphate end may be converted to a 3′ hydroxyl end by a treatment with a chemical or an enzyme, such as alkaline phosphatase, which is routinely carried out by those skilled in the art.

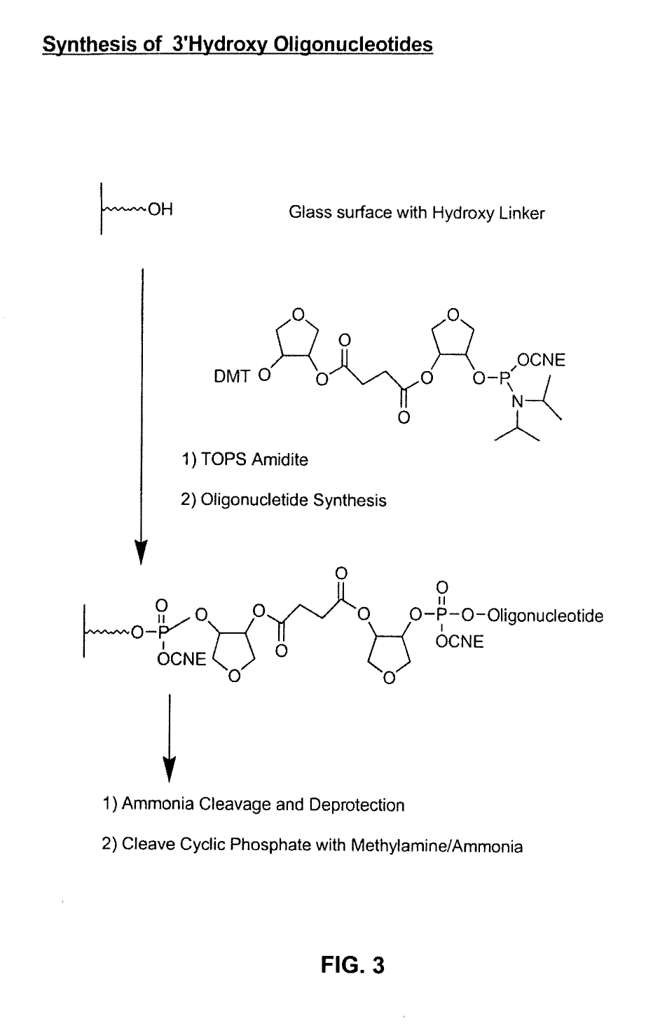

Another class of cleavable linkers is described by McLean, et al. in PCT publication no. WO 93/20092, incorporated herein by reference. This class of cleavable linker, also known as TOPS for two oligonucleotides per synthesis, was designed for generating two oligonucleotides per synthesis by first synthesizing an oligonucleotide on a solid support, attaching the cleavable TOPS linker to the first oligonucleotide, synthesizing a second oligonucleotide on the TOPS linker, and finally cleaving the linker from both the first and second oligonucleotides. In the present invention however, the TOPS phosphoramidite may be used to convert a non-cleavable hydroxyl group on the solid support to a cleavable linker, suitable for synthesizing a large number of overlapping oligonucleotides. A preferred embodiment of TOPS reagents is the Universal TOPS™ phosphoramidite, which is shown in FIG. 3. The conditions for Universal TOPS™ phosphoramidite preparation, coupling and cleavage are detailed in Hardy et al., Nucleic Acids Research 22(15):2998-3004 (1994), which is incorporated herein by reference. The Universal TOPS™ phosphoramidite yields a cyclic 3′ phosphate that may be removed under basic conditions, such as the extended amonia and/or ammonia/methylamine treatment, resulting in the natural 3′ hydroxy oligonucleotide.

A cleavable amino linker may also be employed in the synthesis of overlapping oligonucleotides. The resulting oligonucleotides bound to the linker via a phosphoramidite linkage may be cleaved with 80% acetic acid yielding a 3′-phosphorylated oligonucleotide.

Determination of Overlapping Oligonucleotides Encoding the Long DNA Sequence of Interest

The present method represents a general method for synthesizing and assembling any long DNA sequence from an array of overlapping oligonucleotides. Preferably, the size of the long DNA region ranges from 200 to 10,000 bases. More preferably, the size of the long DNA region ranges from 400 to 5,000 bases. The long DNA sequence of interest may be split into a series of overlapping oligonucleotides. With the enzymatic assembly of the long DNA sequence, it is not necessary that every base, both the sense and antisense strand, of the long DNA sequence of interest be synthesized. The overlapping oligonucleotides are typically required to collectively encode both strands of the DNA region of interest. The length of each overlapping oligonucleotide and the extent of the overlap may vary depending on the methods and conditions of oligonucleotide synthesis and assembly. Several general factors may be considered, for example, the costs and errors associated with synthesizing modest size oligonucleotides, the annealing temperature and ionic strength of the overlapping oligonucleotides, the formation of unique overlaps and the minimization of non-specific binding and intramolecular base pairing, among others. Although, in principle, there is no inherent limitation to the number of overlapping oligonucleotides that may be employed to assemble them in to the target sequence, the number of overlapping oligonucleotides is preferably from 10 to 10,000, and more preferably, from 100 to 5,000.

In particular, for the assembly method using self-priming PCR (infra), a unique overlap is preferred in order to produce the correct size of long DNA sequence after assembly. Unique overlaps may be achieved by increasing the degree of overlap. However, increasing the degree of overlap adds the number of bases required, which naturally incurs additional cost in oligonucleotide synthesis. Those skilled in the art will select the optimal length of the overlapping oligonucleotides and the optimal length of the overlap suitable for oligonucleotide synthesis and assembly methods. In particular, a computer search of both strands of the target sequence with the sequences of each of the overlap regions may be used to show unique design of oligonucleotides with the least likelihood of give nonspecific binding. Preferably, the length of each oligonucleotide may be in the range of about 10 to 200 bases long. More preferably, the length of each oligonucleotide is in the range of 30 to 100 bases long. Preferably, oligonucleotides overlap their complements by about 10 to 100 bases. The lowest end of the range, at least a 10-base overlap, is necessary to create stable priming of the polymerase extension of each strand. At the upper end, maximally overlapped oligonucleotides of 200 bases long would contain 100 bases of complementary overlap. Most preferably, the overlapping regions, in the range of 15-20 base pairs in length may be designed to give a desired melting temperatures, typically in the range of 52-56° C., which ensure primer specificity. It may also be preferred that all overlapping oligonucleotides have a similar extent of overlap and thus a similar annealing temperature, which will normalize the annealing conditions during PCR cycles.

Oligonucleotide Synthesis

Synthesis of oligonucleotides may be best accomplished using a variety of chip or microarray based oligonucleotide synthesis methods. Traditional solid phase oligonucleotide synthesis on controlled-pore glass may also be employed, in particular when the number of oligonucleotides required to assemble the desired DNA sequence is small. Oligonucleotides may be synthesized on an automated DNA synthesizer, for example, on an Applied Biosystems 380A synthesizer using 5-dimethoxytritylnucleoside β-cyanoethyl phosphoramidites. Synthesis may be carried out on a 0.2 μM scale CPG solid support with an average pore size of 1000 Å. Oligonucleotides may be purified by gel electrophoresis, HPLC, or other suitable methods known in the art.

In the preferred embodiment of the instant invention, oligonucleotide synthesis may be performed on a patterned chip using a piezoelectric pump to deliver reagents. The method for conducting an array of oligonucleotide synthesis using a piezoelectric pump is described in U.S. Pat. No. 5,474,796, incorporated herein by reference. This pump delivers microdroplets of chemical reactants to spots separated from the others by surface tension. Typically the support surface has 10-10,000 functional binding sites per cm² and each functionalized binding site is 50-2000 microns in diameter. Typically the amounts of reagents added to each site is in a volume of about 50 picoliters to 2 microliters.

The array plates are made by coating a support surface with a positive or negative photoresist substance. Photoresist substances are readily known to those of skill in the art. For example, an optical positive photoresist substance, for example, AZ 1350 (Novolac™ type-Hoechst Celanese™, Novolac™ is a proprietary novolak resin, which is the reaction product of phenols with formaldehyde in an acid condensation medium), or an E-beam positive photoresist substance, for example, EB-9 (polymethacrylate by Hoya™) may be used.

The photoresist substance coated surface is subsequently exposed and developed to create a patterned region of a first exposed support surface. The exposed surface is then reacted with a fluoroalkylsilane to form a stable hydrophobic matrix. The remaining photoresist is then removed and the glass chip reacted with an amonisilane or hydroxy group bearing silane to form hydrophilic spots. Alternatively, the patterned support surface may be made by reacting a support surface with a hydroxy or aminoalkylsilane to form a derivatized hydrophilic support surface. The support surface is then reacted with o-nitrobenzyl carbonyl chloride as a temporary photolabile blocking to provide a photoblocked support surface. The photoblocked support surface is then exposed to light through a mask to create unblocked areas on the support surface with unblocked hydroxy or aminoalkylsilane. The exposed surface is then reacted with perfluoroalkanoyl halide or perfluoroalkylsulfonyl halide to form a stable hydrophobic (perfluoroacyl or perfluoroalkylsulfonamido) alkyl siloxane matrix. This remaining photoblocked support surface is finally exposed to create patterned regions of the unblocked hydroxy or aminoalkylsilane to form the derivatized hydrophilic binding site regions. A number of siloxane functionalizing reagents may be used. For example, hydroxyalkyl siloxanes, diol (dihydroxyalkyl) siloxanes, aminoalkyl siloxanes, and dimeric secondary aminoalkyl siloxanes, may be used to derive the patterned hydrophilic hydroxyl or amino regions.

A number of support surfaces may be used in array oligonucleotide synthesis using a piezoelectric pump. There are two important characteristics of the masked surfaces in patterned oligonucleotide synthesis. First, the masked surface must be inert to the conditions of ordinary oligonucleotide synthesis. The solid surface must present no free hydroxy or amino groups to the bulk solvent interface. Second, the surface must be poorly wet by common organic solvents such as acetonitrile and the glycol ethers, relative to the more polar functionalized binding sites. The wetting phenomenon is a measure of the surface tension or attractive forces between molecules at a solid-liquid interface, and is defined in dynes/cm². Fluorocarbons have very low surface tension because of the unique polarity (electronegativity) of the carbon-flourine bond. In tightly structured Langmuir-Blodgett type films, surface tension of a layer is primarily determined by the percent of fluorine in the terminus of the alkyl chains. For tightly ordered films, a single terminal trifluoromethyl group will render a surface nearly as lipophobic as a perfluoroalkyl layer. When fluorocarbons are covalently attached to an underlying derivatized solid (highly crosslinked polymeric) support, the density of reactive sites will generally be lower than Langmuir-Blodgett and group density. However, the use of perfluoroalkyl masking agents preserves a relatively high fluorine content in the solvent accessible region of the supporting surface.

Glass (polytetrasiloxane) are particularly suitable for patterned oligonucleotide synthesis using a piezoelectric pump to deliver reagents, because of the numerous techniques developed by the semiconductor industry using thick films (1-5 microns) of photoresists to generate masked patterns of exposed glass surfaces. The first exposed glass surface may be derivatized preferably with volatile fluoroalkyl silanes using gas phase diffusion to create closely packed lipophobic monolayers. The polymerized photoresist provides an effectively impermeable barrier to the gaseous fluoroalkyl silane during the time period of derivatization of the exposed region. Following lipophobic derivatization however, the remaining photoresist can be readily removed by dissolution in warm, organic solvents (methyl, isobutyl, ketone, or N-methyl pyrrolidone) to expose a second surface of raw glass, while leaving the first applied silane layer intact. This second region glass may then be derivatized by either solution or gas phase methods with a second, polar silane which contains either a hydroxyl or amino group suitable for anchoring solid phase oligonucleotide synthesis. Siloxanes have somewhat limited stability under strongly alkaline conditions.

A number of organic polymers also have desirable characteristics for patterned oligonucleotide synthesis. For example, Teflon (polytetrafluoroethylene) may provide an ideal lipophobic surface. Patterned derivatization of this type of material may be accomplished by reactive ion or plasma etching through a physical mask or using an electron beam, followed by reduction to surface hydroxymethyl groups. Polypropylene/polyehtylene may be surface derivatized by gamma irradiation or chromic acid oxidation, and converted to hydroxy or aminomethylated surfaces. Highly crosslinked polystryene-divinylbenzene (ca. 50%) is non-swellable, and may be readily surface derivatized by chloromethlylation and subsequently converted to other functional groups. Nylon provides an initial surface of hexylamino groups, which are directly active. The lipophobic patterning of these surfaces may be effected using the same type of solution-based thin film masking techniques and gas phase derivatization as glass, or by direct photochemical patterning using o-nitrobenzylcarbonyl blocking groups. Perfluoroalkyl carboxylic and sulfonic acid derivatives rather than silanes are now used to provide the lipophobic mask of the underlying surface during oligonucleotide synthesis. Subsequent to the patterning of these surfaces, suitable cleavable linkers are coupled to the reactive group such as the hydroxy or amino group before the addition of nucleoside phosphoramidite.

The solution of chemical reactant may be added to the functionalized binding site through utilization of a piezoelectric pump in an amount where the solution of chemical reactant at each binding site is separate from the solution of chemical reactant at other binding sites by surface tension. The design, construction, and mechanism of a piezoelectric pump are described in Brennan, U.S. Pat. No. 5,474,796, which is incorporated herein by reference. A piezoelectric pump that may be utilized in the instant invention delivers minute droplets of liquid to a surface in a very precise manner. The pump design is similar to the pumps used in ink jet printing. The picopump is capable of producing 50 micron or 65 picoliter droplets at up to 3000 Hz and can accurately hit a 250 micron target in a 900° C. oven at a distance of 2 cm in a draft free environment.

After derivatizing the initial silane as described above, assembly of oligonucleotides on the prepared dots is carried out according to the phosphoramidite method. Acetonitrile is replaced by a high-boiling mixture of adiponitrile, N-methyl-pyrrolidone and acetonitrile (4:1:1) in order to prevent evaporation of the solvent on an open glass surface. Delivery of the blocked phosphoramidites and the activator (S-ethyl-tetrazole) is directed to individual spots using a picopump apparatus. All other steps including detritylation, capping, oxidation and washing, are performed on the array in a batch process by flooding the surface with the appropriate reagents.

Upon the completion of synthesis, the oligonucleotides are cleaved from the surface by standard deprotection procedures, such as ammonia or methylamine/ammonia treatment. The resulting mixture of deprotected oligonucleotides may be directly used for assembly or PCR without further purification.

Assembly of the Mixture of Overlapping Oligonucleotides into the Full-Length Target Sequence

Assembly of the target long DNA sequence from a series of overlapping oligonucleotides may be accomplished using a variety of methods in the literature known to those skilled in the art. The standard approach is to use enzymatic ligation to arrive at the target DNA of the desired length. The overlapping oligonucleotides may be annealed to form double-strand DNA sequence with single stranded and/or double-stranded breaks. These breaks may then be filled in with a DNA polymerase and/or enzymatically ligated using DNA ligases using known methods in the art. For example, T4 DNA ligase may be used to ligate two blunt end oligonucleotides. Intermolecular ligation of the 5′ and 3′ ends of oligonucleotides through the formation of a phosphodiester bond typically requires one oligonucleotide bearing a 5′-phosporyl donor group and another with a free 3′-hydroxyl acceptor. Oligonucleotides may be phosphorylated using known methods in the art. The full-length target DNA sequence may then be amplified using PCR-based technology or cloned into a vector of choice using known methods in the art.

1. Assembly Using Oligonucleotide Directed Double-Strand Break Repair.

Oligonucleotide directed double-strand break repair may be employed to assemble short oligonucleotides into long DNA sequences using. Mandecki, Proc. Natl. Acad. Sci. USA 83:7177-7181 (1986); Mandecki et al., Gene 68:101-107 (1988) and Mandecki et al., Gene 94:103-107 (1990). This method comprises of three essential steps: (1) cloning suitable inserts in a plasmid DNA; (2) generating restriction fragments with protruding ends from the insert containing plasmid DNA; and (3) assembling the restriction fragments into the target long DNA sequence.

In preferred embodiments, cloning of inserts in a plasmid DNA in step 1 may be carried out using oligonucleotide directed double-strand break repair, also known as the bridge mutagenesis protocol. The oligonucleotide directed double-strand break repair essentially involves the transformation of E. coli with a denatured mixture of one or more oligonucleotide inserts and a linearized plasmid DNA, wherein the 5′ and 3′ ends of the oligonucleotide inserts are homologous to sequences flanking the double-strand break (i.e., the cleavage site) of the linearized plasmid DNA. The homologous sequences at the 5′ and 3′ ends of the oligonucleotide inserts direct, in vivo, the repair of the double-strand break of the linearized plasmid DNA. The homologous sequence between each side of the double-strand break and each end of the oligonucleotide insert is typically more than 10 nt long. In preferred embodiments, the homologous sequences at the 5′ and 3′ end of oligonucleotides and the two sides of the double-strand break contain FokI recognition sites (5′-GGATG-3′). A series of overlapping subsequences of the target DNA sequence are inserted between two FokI sites.

The FokI restriction enzyme creates a staggered double-strand break at a DNA site 9 and 13 nt away from its recognition site, which upon cleavage of the plasmid DNA with FokI, a restriction is liberated that contains unique 4 nt long 5′ protruding ends. The uniqueness of ends permits efficient and direct simultaneous ligation of the restriction fragments to form the target long DNA sequence. The oligonucleotide inserts using the FokI method of gene assembly are designed by dividing the target long DNA sequence into a series of subsequences of clonable size, typically in the range of 20 nt to 200 nt. The division points may be preferably between the codons of the open reading frame (ORF) of the target long DNA sequence. Each subsequence may overlap its neighboring subsequence on either side by four nt, so that the overlapping regions will form complementary cohesive ends when the cloned subsequences are removed from the plasmid DNA with FokI restriction enzyme. In particular, the overlapping subsequences are typically chosen such that they are unique, which will assure that they may be annealed to each other in the proper arrangement during the assembly of subsequences into the target long DNA sequence following the FokI cleavage. In particular, if there is any FokI site within the target DNA sequence, it may be preferably placed within an overlap region, which causes FokI cleavage at this site to fall outside the cloned regions. Once the subsequences containing the four nt overlap have been determined, sequences of the oligonucleotide inserts may be obtained by adding two arms to provide the necessary sequence homology to two sides of the double-strand break of the DNA plasmid. The sequence of oligonucleotide inserts thus take the form of arm1+subsequence (containing 4 nt overlap)+arm2, in which arm1 and arm2, each containing the FokI site, are homologous to the respective side of the double-strand break of the DNA plasmid. The total length of the oligonucleotide inserts may be varied to optimize the efficiency of break repair. In addition, position of the subsequence with respect to the homologous sites may also be varied to optimize the efficiency of repair. It is known that the efficiency of repair decreases as the distance between the subsequence and the homologous sequence increases. In particularly preferred embodiments using the FokI method of gene assembly, the average length of a subsequence is about 70 nt and arm1 and arm2 of the oligonucleotide inserts are 15 nt each, containing the FokI site and sequences complementary to sequences flanking the double-strand break of the plasmid DNA. Therefore, in particularly preferred embodiments, the average length of the oligonucleotide insert is 100 nt (arm1+subsequence+arm2).

The oligonucleotide inserts may then be cloned into a suitable vector by the bridge mutagenesis protocol. A suitable plasmid system may be selected based on the existence of a cluster of unique restriction sites for cleaving plasmid DNA and the feasibility of an easy and accurate screening method for the insert containing colonies of plasmid. A color screening method is particularly preferred. The pUC plasmid, which contains multiple cloning sites and an indicator gene (the lacZ gene or a fragment thereof) may be used. In particular, a frame shift mutation may be introduced to the multiple cloning site of pUC in order to effect a suitable screening method. For example, a deletion of one residue at the PstI site may be introduced to the pUC plasmid. The oligonucleotide insert introduced into the mutated plasmid may then contain one extra nucleotide to restore the reading frame of the lacZ when the repair of the double-strand break of the plasmid DNA occurs. This way, the insert containing plasmids are readily selected, as the repaired plasmid (or the insert containing) form blue colonies, while the cells containing the nucleotide deletion (the parent) plasmid gives rise to white colonies. It is also advantageous that all insertions introduced into plasmid are designed such that they would destroy a unique restriction site within the multiple cloning sites and at the same time create a new restriction site. This feature would allow for an additional confirmation of the insertion event by restriction digestion of plasmid DNA. Suitable DNA plasmid used for the FokI method of gene assembly may be cut with a restriction enzyme, preferably a unique restriction site. While it is convenient that the linearized plasmid is obtained by restriction enzyme cleavage at one site, the present invention also contemplates other methods for generating the linearized plasmid DNA, such as, by restriction enzyme cleavage at multiple sites, reconstructing a linear plasmid by ligating DNA fragments, or random cleavage of DNA using DNase digestion, sonication, among others. The linearized DNA plasmid may be mixed with the oligonucleotide inserts under denaturing conditions and the mixture may be transformed into a suitable organism, such as E. coli with suitable compotent cells using known methods in the art. Conditions may be varied to improve the efficiency of repair, for example, the molar ratio of oligonucleotide inserts and the plasmid DNA, the denaturing conditions, among others. Typically, a molar excess of oligonucleotide over plasmid DNA is necessary for efficient repair of the double-strand, typically in the range from 10-fold to 1000-fold molar excess of oligonucleotide inserts. It may also be necessary to denature the linear plasmid DNA before using transformation, for example by incubating the mixture of plasmid NDA and oligonucleotide inserts at 100° C. for 3 min. Plasmid constructs containing the FokI fragments may be selected using the designed screening method.

The insert containing DNA plasmid may then be digested with FokI (New England BioLabs) under the conditions recommended by the manufacture. The FokI fragments may then be purified and joined together in a single ligation reaction according to a standard protocol known in the art. The FokI fragments of the oligonucleotide inserts contain subsequences with unique complementary 4-bp overhangs which, when annealed and ligated, formed the target long DNA sequence. It should be noted that the ligation of FokI restriction fragment is not limited to DNA fragments introduced by the bridge mutagenesis. Protruding ends with 4 nt 5′ overhangs may be generated by other methods, for example, by FokI digestion of any DNA sequence. Successful assembly of the target long DNA sequence may be verified by DNA sequencing, hybridization-based diagnostic method, molecular biology techniques, such as restriction digest, selection marker, or other suitable methods. DNA manipulations and enzyme treatments are carried out in accordance with established protocols in the art and manufacturers' recommended procedures. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985).

Assembly method using oligonucleotide directed double-strand break repair is particularly flexible where it does not require the presence of any restriction sites within the target DNA sequence. The cost of this method is low because of the reduction in the total length of synthetic oligonucleotide needed to construct the target long DNA sequence. Only one DNA strand of the target DNA sequence needs to be obtained synthetically, compared to conventional methods where both DNA strands are made synthetically. The method is accurate with low frequency of sequence error, because the in vivo double-strand repair rather than the in vitro ligation allows for a biological selection of the unmodified oligonucleotides and the subsequent screening of insert containing colonies further eliminates the undesired recombination products.

2. Assembly Using Self-Priming Pcr.

Self-priming PCR may also be used as a method for assembling short overlapping oligonucleotides into long DNA sequences. See Dillon et al., BioTechniques 9(3):298-300 (1990), Hayashi et al., BioTechniques 17(2):310-315 (1994), Chen et al., J. Am. Chem. Soc. 116:8799-8800 (1994), and Prodromou et al, Protein Engineering 5(8):827-829 (1992). Essentially, overlapping oligonucleotides, which collectively represent the target long DNA sequence, are mixed and subjected to PCR reactions, such that those overlapping at their 3′ ends are extended to give longer double-strand products and repeated until the full-sized target sequence is obtained.

The overlapping oligonucleotides may be mixed in a standard PCR containing dNTP, DNA polymerase of choice, and buffer. The overlapping ends of the oligonucleotides, upon annealing, create short regions of double-strand DNA and serve as primers for the elongation by DNA polymerase in a PCR reaction. Products of the elongation reaction serve as substrates for formation of a longer double-strand DNA, eventually resulting in the synthesis of full-length target sequence. The PCR conditions may be optimized to increase the yield of the target long DNA sequence. The choice of the DNA polymerase for the PCR reactions is based on its properties. For example, thermostable polymerases, such as Taq polymerase may be used. In addition, Vent DNA polymerase may be chosen in preference to Taq polymerase because it possesses a 3′-5′ proofreading activity, a strand displacement activity and a much lower terminal transferase activity, all of which serve to improve the efficiency and fidelity of the PCR reactions.

Although it is possible to obtain the target sequence in a single step by mixing all the overlapping oligonucleotides, PCR reactions may also be performed in multiple steps, such that larger sequences might be assembled from a series of separate PCR reactions whose products are mixed and subjected to a second round of PCR. For example, it has been shown that the addition of 5′ and 3′ primers at the end of first round PCR reactions may be advantageous to generate the full-length DNA product. In other instances, additional sequences, such as restriction sites, a Shine-Dalgamo sequence, and a transcription terminator, among other, may be desirably added to the target sequence to facilitate the subsequent cloning of the target sequence gene into expression vectors. These new sequences may require additional primers and additional PCR reactions. Moreover, if the self-priming PCR fails to give a full-sized product from a single reaction, the assembly may be rescued by separately PCR-amplifying pairs of overlapping oligonucleotides, or smaller sections of the target DNA sequence, or by the conventional filling-in and ligation method.

Successful assembly of the target long DNA sequence may be verified by DNA sequencing, hybridization-based diagnostic method, molecular biology techniques, such as restriction digest, selection marker, or other suitable methods. DNA manipulations and enzyme treatments are carried out in accordance with established protocols in the art and manufacturers' recommended procedures. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985).

There are several advantages to the assembly method using self-priming PCR. It generally requires neither phosphorylation nor ligation, while giving high yields. The cost of this method is relatively low because it reduces the number of oligonucleotides needed for synthetic constructions. Only oligonucleotides representing the partial sequence of each strand are synthesized and the gaps in the annealed oligonucleotides are filled in using DNA polymerase during PCR. The assembly of overlapping oligonucleotides may be achieved in a one-pot single step PCR with no requirement for isolation and purification of intermediate products. In particular, gel purification of oligonucleotides are not necessary and crude oligonucleotide preparations may be directly used for PCR. Furthermore, this method of assembly is accurate and does not require the existence of restriction enzyme sites in the target sequence.

### EXAMPLES

The following examples further illustrate the present invention. These examples are intended merely to be illustrative of the present invention and are not to be construed as being limiting. The examples are intended specifically to illustrate recoveries of virus, protein and peptide of interest which may be attained using the process within the scope of the present invention.

### Example 1

Preparation of Array Plates Ready for Oligonucleotide or Peptide Assembly

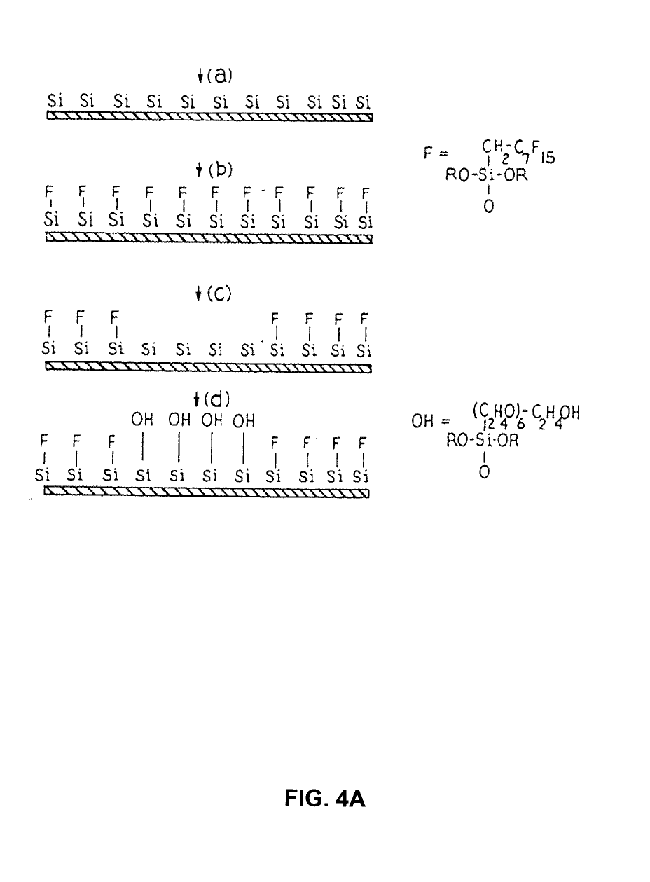

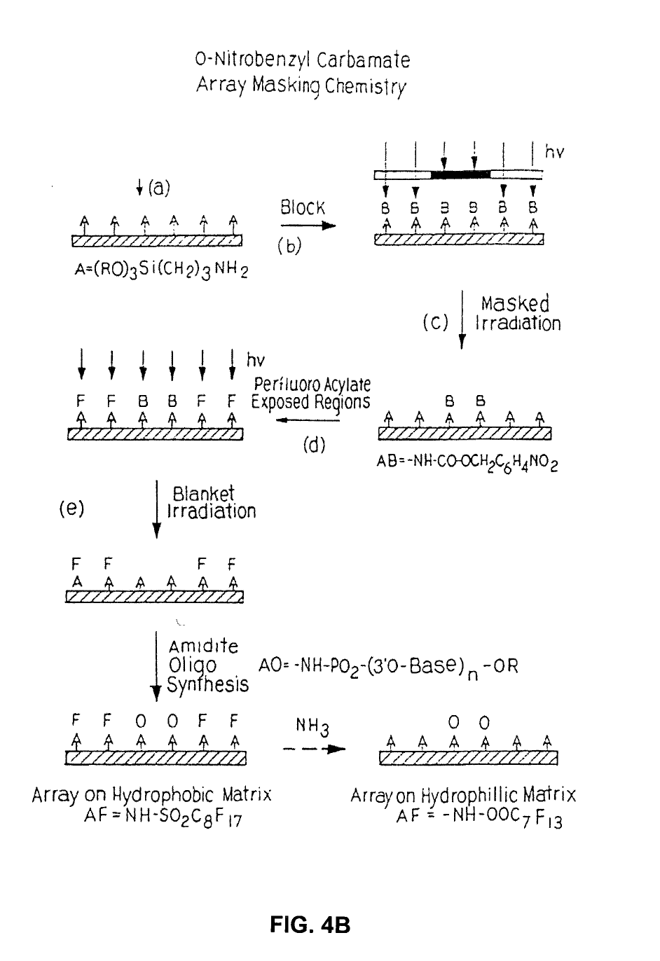

The hybridization array is synthesized on a glass plate. The plate is first coated with the stable fluorosiloxane 3-(1,1-dihydroperfluoroctyloxy) propyltriethoxysilane. A CO₂ laser is used to ablate off regions of the fluorosiloxane and expose the underlying silicon dioxide glass. The plate is then coated with glycidyloxypropyl trimethoxysilane, which reacts only on the exposed regions of the glass to form a glycidyl epoxide. The plate is next treated with hexaethyleneglycol and sulfuric acid to convert the glycidyl epoxide into a hydroxyalkyl group, which acts as a linker arm. The hydroxyalkyl group resembles the 5′-hydroxide of nucleotides and provides a stable anchor on which to initiate solid phase synthesis. The hydroxyalkyl linker arm provides an average distance of 3-4 nm between the oligonucleotide and the glass surface. The siloxane linkage to the glass is completely stable to all acidic and basic deblocking conditions typically used in oligonucleotide or peptide synthesis. This scheme for preparing array plates is illustrated in FIGS. 4A and 4B and was previously discussed.

### Example 2

Assembly of Oligonucleotides on the Array Plates

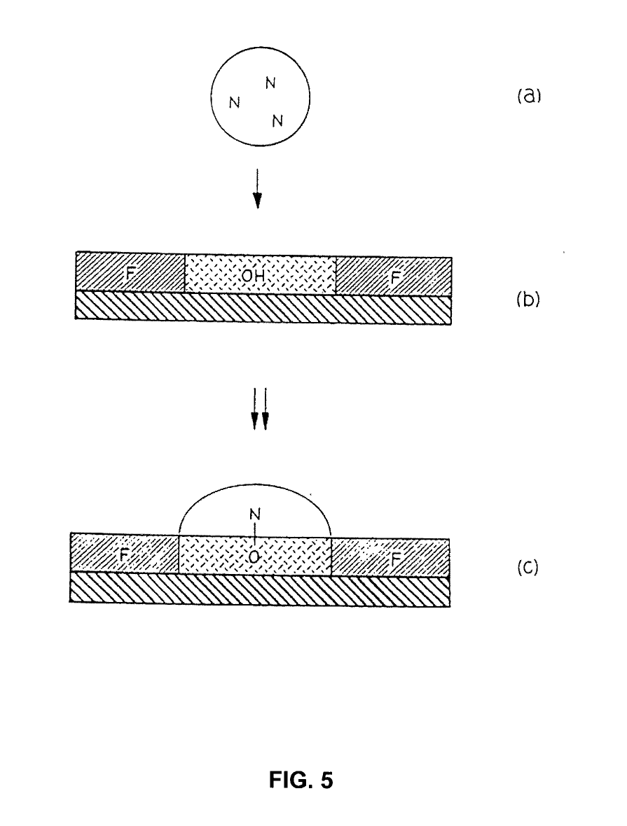

The hydroxyalkylsiloxane surface in the dots has a surface tension of approximately γ=47, whereas the fluoroxysilane has a surface tension of γ=18. For oligonucleotide assembly, the solvents of choice are acetonitrile, which has a surface tension of γ=29, and diethylglycol dimethyl ether. The hydroxyalkylsiloxane surface is thus completely wet by acetonitrile, while the fluorosiloxane masked surface between the dots is very poorly wet by acetonitrile. Droplets of oligonucleotide synthesis reagents in acetonitrile are applied to the dot surfaces and tend to bead up, as shown in FIG. 5. Mixing between adjacent dots is prevented by the very hydrophobic barrier of the mask. The contact angle for acetonitrile at the mask-dot interface is approximately θ=43°. The plate effectively acts as an array microliter dish, wherein the individual wells are defined by surface tension rather than gravity. The volume of a 40 micron droplet is 33 picoliter. The maximum volume retained by a 50 micron dot is approximately 100 picoliter, or about 3 droplets. A 100 micron dot retains approximately 400 picoliter, or about 12 droplets. At maximum loading, 50 micron and 100 micron dots bind about 0.07 and 0.27 femtomoles oligonucleotide, respectively.

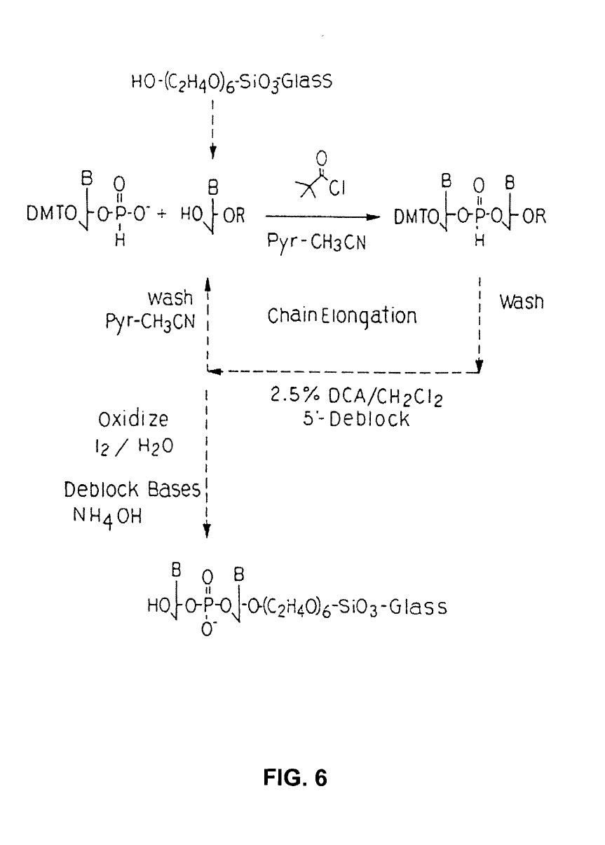

Assembly of oligonucleotides on the prepared dots (FIG. 4B, bottom) is carried out according to the H-phosphonate procedure (FIG. 6), or by the phosphoroamidite method. Both methods are well known to those of ordinary skill in the art. Christodoulou, C., “Oligonucleotide Synthesis” in “Protocols for oligonucleotides and analogs; synthesis and properties,” Methods Mol. Biol. 20:19-31 (1993). Beaucage, S., “Oligodeoxyribonucleotides Synthesis” in “Protocols for oligonucleotides and analogs; synthesis and properties,” Methods Mol. Biol. 20:33-61 (1993). Delivery of the appropriate blocked nucleotides and activating agents in acetonitrile is directed to individual dots using the picopump apparatus described in Example 3. All other steps, (e.g., DMT deblocking, washing) are performed on the array in a batch process by flooding the surface with the appropriate reagents. An eight nozzle piezoelectric pump head is used to deliver the blocked nucleotides and activating reagents to the individual dots, and delivering droplets at 1000 Hz, requires only 32 seconds to lay down a 512×512 (262 k) array. Since none of the coupling steps have critical time requirements, the difference in reaction time between the first and lost droplet applied is insignificant.

### Example 3

Construction of Piezoelectric Impulse Jet Pump Apparatus

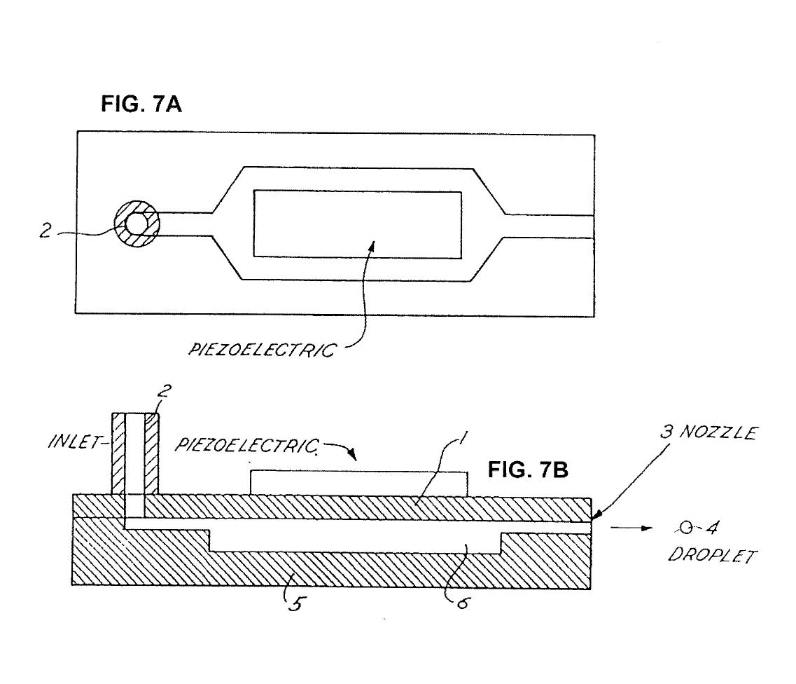

Piezoelectric impulse jets are fabricated from Photoceram (Corning Glass, Corning, N.Y.), a UV sensitive ceramic, using standard photolithographic techniques to produce the pump details. The ceramic is fired to convert it to a glassy state. The resulting blank is then etched by hydrogen fluoride, which acts faster in exposed then in nonexposed areas. After the cavity and nozzle details are lapped to the appropriate thickness in one plate, the completed chamber is formed by diffusion bonding a second (top) plate to the first plate. The nozzle face is lapped flat and surface treated, then the piezoelectric element is epoxied to the outside of the pumping chamber. When the piezoelectric element is energized it deforms the cavity much like a one-sided bellows, as shown in FIG. 7.

To determine the appropriate orifice size for accurate firing of acetonitrile droplets, a jet head with a series of decreasing orifice sizes is prepared and tested. A 40 micron nozzle produces droplets of about 65 picoliter.

A separate nozzle array head is provided for each of the four nucleotides and a fifth head is provided to deliver the activating reagent for coupling. The five heads are stacked together with a mechanically defined spacing. Each head has an array of eight nozzles with a separation of 400 microns.

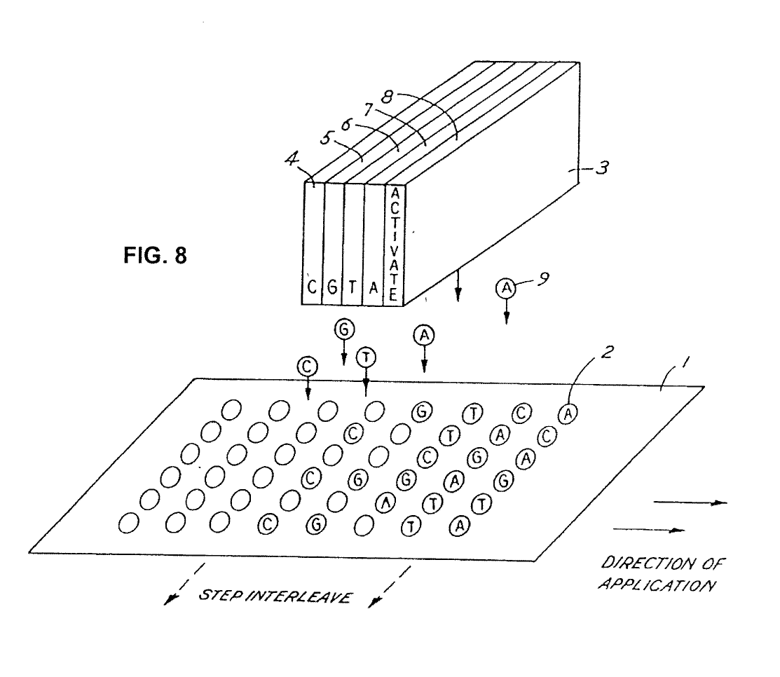

The completed pump unit is assembled with the heads held stationary and the droplets fired downward at a moving array plate as shown in FIG. 8. The completed pump unit assembly (3) consists of nozzle array heads (4-7) for each of the four nucleotidase and a fifth head (8) for activating reagent. When energized, a microdroplet (9) is ejected from the pump nozzle and deposited on the array plate (1) at a functionalized binding site (2).

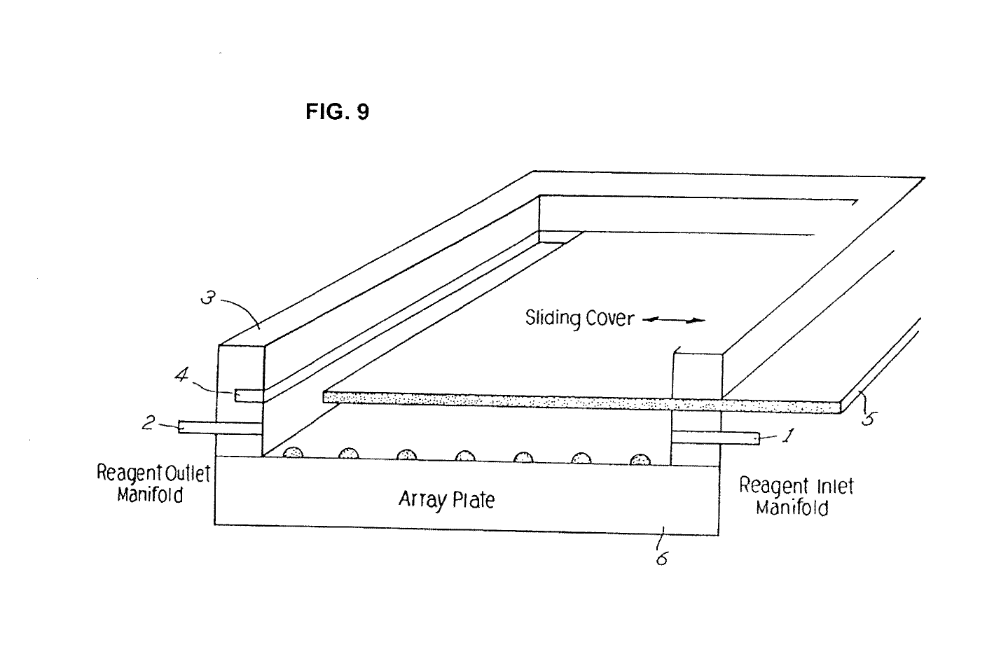

A plate holding the target array is held in a mechanical stage and is indexed in the X and Y planes beneath the heads by a synchronous screw drives. The mechanical stage is similar to those used in small milling machines, microscopes and microtomes, and provides reproducible positioning accuracy better than 2.5 microns or 0.1 mil. As shown in FIG. 9, the plate holder (3) is fitted with a slotted spacer (4) which permits a cover plate (5) to be slid over the array (6) to form an enclosed chamber. Peripheral inlet (1) and outlet (2) ports are provided to allow the plate to be flooded for washing, application of reagents for a common array reaction, or blowing the plate dry for the next dot array application cycle.

Both the stage and head assembly are enclosed in a glove box which can be evacuated or purged with argon to maintain anhydrous conditions. With the plate holder slid out of the way, the inlet lines to the heads can be pressurized for positive displacement priming of the head chambers or flushing with clean solvent. During operation, the reagent vials are maintained at the ambient pressure of the box.

With a six minute chemistry cycle time, the apparatus can produce 10-mer array plates at the rate of 1 plate or 10⁶ oligonucleotides per hour.

Although the invention has been described with reference to the presently preferred embodiments, it should be understood that various modifications can be made without departing from the spirit of the invention.

We claim:

1. A method for producing a biologically functional DNA sequence of greater than 200 bases long comprising the steps of:
(a) synthesizing on a substrate an array of overlapping oligonucleotides from 10 to 200 bases encoding for either sense or antisense strand of said biologically functional DNA sequence wherein said oligonucleotides are covalently attached to the substrate using a cleavable linker;
(b) cleaving said oligonucleotides from the substrate; and
(c) assembling the mixture of overlapping oligonucleotides into said biologically functional DNA sequence.

2. The method according to claim 1 wherein said overlapping oligonucleotides are from 30 to 100 bases long.

3. The method according to claim 1 wherein the length of said biologically functional DNA sequence ranges from 200 to 10,000 bases.

4. The method according to claim 3 wherein the length of said biologically functional DNA sequence ranges from 400 to 5,000 bases.

5. The method according to claim 1 wherein said cleavable linker is a succinate like compound.

6. The method according to claim 1 wherein the number of overlapping oligonucleotides in the array is from 10 to 10,000.

7. The method according to claim 6 wherein the number of overlapping oligonucleotides in the array is from 100 to 5,000.

8. The method according to claim 1 wherein assembling the mixture of oligonucleotides further comprising enzymatic ligation.

9. The method according to claim 1 wherein assembling the mixture of oligonucleotides further comprising PCR technology.

10. The method according to claim 1 wherein assembling the mixture of oligonucleotides further comprising hybridization.

11. The method according to claim 1 wherein said biologically functional DNA sequence encodes a gene.

12. The method according to claim 1 wherein said biologically functional DNA sequence is a plasmid.

13. The method according to claim 1 wherein said biologically functional DNA sequence is a virus.

14. The method according to claim 1 wherein said biologically functional DNA sequence is the genome of an organism.

15. A biologically functional DNA sequence recovered according to the method of claim 1.

16. A substrate containing a cleavable linker for oligonucleotide synthesis according to the method of claim 1.

17. A method for optimizing the function of a DNA sequence comprising the steps of:
(a) synthesizing on a substrate an array of overlapping oligonucleotides from 10-200 bases encoding for either sense or antisense strand of said DNA sequence wherein said oligonucleotides are covalently attached to the substrate using a cleavable linker;
(b) cleaving said oligonucleotides from the substrate;
(c) assembling the mixture of oligonucleotides into said DNA sequence;
(d) testing the function of said DNA sequence; and
(e) repeating the steps of (a)-(d) by varying said DNA sequence to optimize the function.
